(19) United States
(12) Patent Application Publication
(10) Pub. No.: US 2026/0248616 A1
(43) Pub. Date: Aug. 27, 2026

(54) RECHARGEABLE BATTERY RETENTION IN AN ACTIVE IMPLANTABLE MEDICAL DEVICE

(71) Applicant: Boston Scientific Scimed, Inc., Maple Grove, MN (US)

(72) Inventors: Matthew William Couri, Plymouth, MN (US); Derek John Boettger, Woodbury, MN (US); Henry Koon, Saint Paul, MN (US); John Anders Bostrom, Saint Paul, MN (US)

(21) Appl. No.: 19/543,346

(22) Filed: Feb. 18, 2026

Related U.S. Application Data
(60) Provisional application No. 63/762,380, filed on Feb. 24, 2025.

(51) Int. Cl. A61F 2/26; A61F 2/48

(52) U.S. Cl. CPC A61F 2/26; A61F 2/482; A61F 2250/0013

(57) ABSTRACT

An implantable medical device includes: an inflatable member; a fluid reservoir; and an electronic pump device that is configured to transfer fluid between the inflatable member and the fluid reservoir. The electronic pump device includes: a housing; a rechargeable battery that includes a case and at least one electrode, where the rechargeable battery is configured to supply electrical power to the electronic pump device, and where a shape and a size of the rechargeable battery changes in response to the rechargeable battery being recharged; and a liner configured for securing the rechargeable battery to the housing of the electronic pump device and for preventing the case of the rechargeable battery from contacting the housing of the electronic pump device when the shape and size of the rechargeable battery changes in response to the rechargeable battery being recharged.

### CROSS-REFERENCE TO RELATED APPLICATION

This application claims priority to U.S. Provisional Patent Application No. 63/762,380, filed on Feb. 24, 2025, entitled “RECHARGEABLE BATTERY RETENTION IN AN ACTIVE IMPLANTABLE MEDICAL DEVICE”, the disclosure of which is incorporated by reference herein in its entirety.

### TECHNICAL FIELD

This disclosure relates generally to implantable medical devices and, in particular, to rechargeable battery tension in active implantable medical devices.

### BACKGROUND

Some implantable medical devices have an electronic pump, which, when operated by a user, causes a transfer of fluid between a fluid reservoir and an inflatable member. The electronic pump can be powered by a rechargeable battery that is included within the implantable medical device. When the battery is recharged, it can swell and change its size and shape, which, in some cases, can cause the battery to move within the medical device and potentially to compromise mechanical and electrical connections between the battery and other components of the implantable medical device, which could cause degradation or failure of the device.

### SUMMARY

In some aspects, the techniques described herein relate to an implantable medical device that includes: an inflatable member; a fluid reservoir; and an electronic pump device that is configured to transfer fluid between the inflatable member and the fluid reservoir. The electronic pump device includes: a housing; a rechargeable battery that includes a case and at least one electrode, where the rechargeable battery is configured to supply electrical power to the electronic pump device, and where a shape and a size of the rechargeable battery changes in response to the rechargeable battery being recharged; and a liner configured for securing the rechargeable battery to the housing of the electronic pump device and for preventing the case of the rechargeable battery from contacting the housing of the electronic pump device when the shape and size of the rechargeable battery changes in response to the rechargeable battery being recharged.

Implementations can include one or more of the following features, alone or in any combination with each other.

For example, the case of the rechargeable battery can include a first substantially planer surface and a second substantially planar surface that is parallel to the first surface, where a maximum distance between the first surface and the second surface increases by more than 10% in response to the rechargeable battery being recharged.

In another example, the first substantially planar surface has a first width and a first length and the second substantially planar surface has a second width and a second length, wherein the second width is within 10% of the first width and the second length is within 10% of the first length, and wherein a height of the case between the first substantially planar surface and the second substantially planar surface is less than 50% of the first width.

In another example, the implantable medical device can further include one or more electrodes located on a face of the battery defined by the height of the battery and a width that is substantially parallel to the first and second substantially planar surfaces.

In another example, the one or more electrodes can be located on a midplane between the first and second substantially planar surfaces.

In another example, the liner can include a first perimeter portion configured for receiving a first edge portion of the case along a length of the case, a second perimeter portion configured for receiving a second edge portion of the case along a length of the case, and a third perimeter portion configured for receiving a third edge portion of the case along the width of the case, the third perimeter portion being attached to the first and second perimeter portions.

In another example, the first, second, and third perimeter portions can have a height that is more the maximum distance between the first surface and the second surface that is attained in response to the rechargeable battery being recharged.

In another example, the first, second, and third perimeter portions can have a height that is at least 10% greater than the height of the case.

In another example, the liner can include no portions that contact the first substantially planar surface or the second substantially planar surface.

In another example, the liner further can include a fourth perimeter portion configured for receiving a printed circuit board, where the printed circuit board can include at least one electrical component that is powered by the battery.

In another example, the liner further can include a fifth perimeter portion configured for receiving a fourth edge portion of the case along the width of the case.

In another example, the liner further can include one or more first coupling members that couple the liner to the housing of the electronic pump device.

In another example, each of the one or more first coupling members can include a post that is integral to, and extends from, the liner through a respective hole in the housing.

In another example, the first coupling members include an adhesive material disposed on the post.

In another example, each of the first coupling members includes a rivet.

In another example, the rivet can include a head portion, a neck portion, and a flared portion, and the head portion can contact the housing, and the neck portion can be disposed in the hole.

In another example, the liner further can include one or more second coupling members configured for coupling the liner to a printed circuit board, where the printed circuit board can include at least one electrical component that is powered by the battery, where each of the second coupling member can include a post that is integral to liner and is configured to extend from the liner through a hole in the printed circuit board.

In another example, each of the second coupling members can include an adhesive material disposed on the post.

In another example, each of the second coupling members can include a rivet having a head portion, a neck portion, and a flared portion, where the head portion is configured to contact the printed circuit board, and the neck portion is configured to be disposed in a hole in the printed circuit board.

### BRIEF DESCRIPTION OF THE DRAWINGS

FIG. 1 illustrates an implantable medical device according to an aspect.

FIG. 2A is a schematic diagram of an inflatable penile prosthesis.

FIG. 2B is a schematic diagram of an inflatable artificial urinary tract sphincter.

FIG. 3A is a front perspective view of a rechargeable battery that can be used to power electronic components of an implantable medical device.

FIG. 3B is a rear perspective view of the rechargeable battery.

FIG. 4 is a schematic perspective view of the rechargeable battery when it is received and secured by a liner.

FIG. 5A is a schematic end view of the rechargeable battery when it is received and secured by a liner when the battery is in a normal, unpressurized state.

FIG. 5B is a schematic end view of the rechargeable battery when it is received and secured by a liner when the battery is in an unpressurized state, while the battery is being recharged or just after it has been recharged.

FIG. 6A is a schematic perspective view of a battery received and secured within a battery liner.

FIG. 6B is a schematic perspective view of the battery received and secured within the battery liner, with a printed circuit board being received and attached to the liner.

FIG. 6C is a schematic bottom view of the printed circuit board attached to a housing of an electronic device, where the housing can include a fluidic manifold configured for pumping fluid between a reservoir and an inflatable member.

FIG. 7A is a schematic perspective view of a liner that is configured for receiving a battery.

FIG. 7B is a schematic perspective view of the liner that has received and secured the battery.

FIG. 8A is a schematic perspective view of a liner that is configured for receiving a battery.

FIG. 8B is a schematic top view of the liner that is receiving the battery.

FIG. 8C is a schematic top view of the liner that has received the battery.

FIG. 9A is a schematic exploded view of a multi-part liner that is configured for receiving a battery.

FIG. 9B is a schematic perspective view of the liner that has received the battery.

FIG. 10A is a schematic exploded view of another multi-part liner that is configured for receiving a battery.

FIG. 10B is a schematic perspective view of the liner that has received the battery.

FIG. 11A is an example partial side view of a battery disposed within a liner with the liner being attached to a housing of an implantable medical device by a coupling member.

FIG. 11B is another example partial side view of the battery disposed within the liner with the liner being attached to the housing of the implantable medical device by the coupling member.

FIG. 11C is another example partial side view of the battery disposed within the liner with the liner being attached to the housing of the implantable medical device by the coupling member.

FIG. 12A is a schematic exploded view of another multi-part liner that is configured for receiving a battery and for attaching to a housing of an implantable medical device.

FIG. 12B is a schematic perspective view of the liner that has received the battery.

FIG. 13A is a schematic bottom view of a housing of an implantable medical device.

FIG. 13B is a schematic bottom view of the housing with a battery liner, which is attached to a printed circuit board, secured to the housing.

### DETAILED DESCRIPTION

This disclosure relates to an implantable medical device with an electronic pump device configured to automatically transfer fluid between a fluid reservoir and an inflatable member. In some examples, the implantable medical device includes a penile prosthesis with one or more inflatable cylinders. In some examples, the implantable medical device includes a urinary control device with an inflatable cuff. The electronic pump device includes a housing with a fluidic manifold having one or more pumps and one or more valves to facilitate the transfer of fluid between the fluid reservoir and the inflatable member. The implantable medical device includes a rechargeable battery that powers the one or more pumps in the one or more valves. The rechargeable battery is secured to a housing of the implantable medical device by a battery liner that holds the rechargeable battery in place and allows the rechargeable battery to change its shape and size as a result of being recharged. The way the battery liner holds the rechargeable battery in place prevents the rechargeable battery from contacting a housing of the implantable medical device in a way that could cause electorates of the rechargeable battery to move relative to the housing as a result of the recharging of the battery.

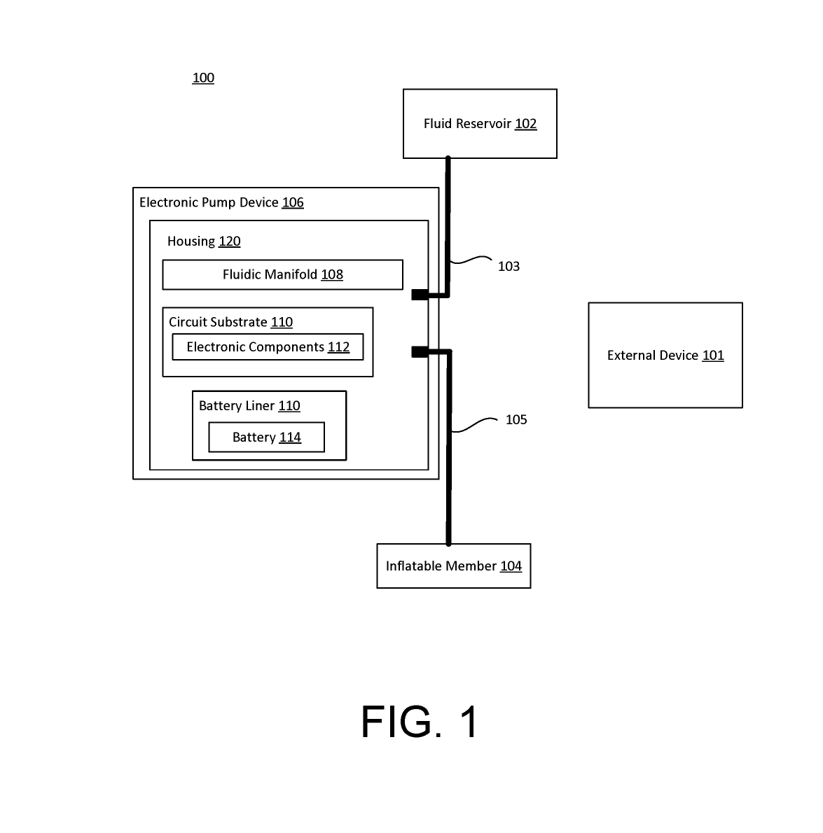

FIG. 1 illustrates an implantable medical device 100 according to an aspect. In some examples, the implantable medical device 100 is an artificial urinary sphincter device. In some examples, the implantable medical device 100 is an inflatable penile prosthesis. However, the implantable medical device 100 may include any type of medical device that includes a rechargeable battery.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user’s abdominal cavity or the upper portion of the user’s pelvic cavity). In some examples, at least a portion of the electronic pump device 106 may be implemented in the patient’s body.

The inflatable member 104 may be capable of expanding upon the injection of fluid into a cavity of the inflatable member 104. If implanted around the urethra, the expansion of the inflatable member 104 causes the urethra to become restricted, thereby

reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 can monitor, control, and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

The electronic pump device 106 includes a housing 120. The housing 120 includes a fluidic manifold 108 that attaches to fluid transfer and pressure regulating components and to a circuit substrate 110 with electronic components 112 and to a battery 114. In some examples, the circuit substrate 110 is a printed circuit board (PCB). The fluidic manifold 108 may include fluidic components such as one or more pumps (e.g., electrically controlled pumps) and one or more valves (e.g., electrically controlled valves).

The electronic pump device 106 includes an energy storage battery (e.g., a rechargeable battery) 114 that powers the electronic components 112. The electronic components 112 can be disposed on a circuit substrate 110, such as a plurality of circuit boards. The battery 114 can assume various forms appropriate to provide power for generating desired electrical signals and to store power provided from the electronic components 112. For example, the battery 114 can incorporate lithium-ion (Li+) chemistry, e.g., a lithium-ion battery to operate the electronic components 112. In some examples, the electronic components 112 can be implemented by various components including resistors, capacitors, transistors, and integrated circuits disposed on the circuit substrate 110.

The electronic components 112 can include a recharge system for recharging the battery 114, a communication system, and a controller. The recharge system includes hardware configured to interface with a coil to receive power signals, and to provide the power signals in a form suitable to recharge the battery 114 and can include circuitry to reduce the likelihood of overcharging the battery 114. The communication system includes hardware configured to interface with an antenna to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices 101 (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna for transmission. In some examples, the communication system can be configured to receive and transmit radio frequency signals via the antenna. The controller can include a microcontroller to operate the recharge system and to receive and operate in response to communication signals or generate communication signals from the communication system.

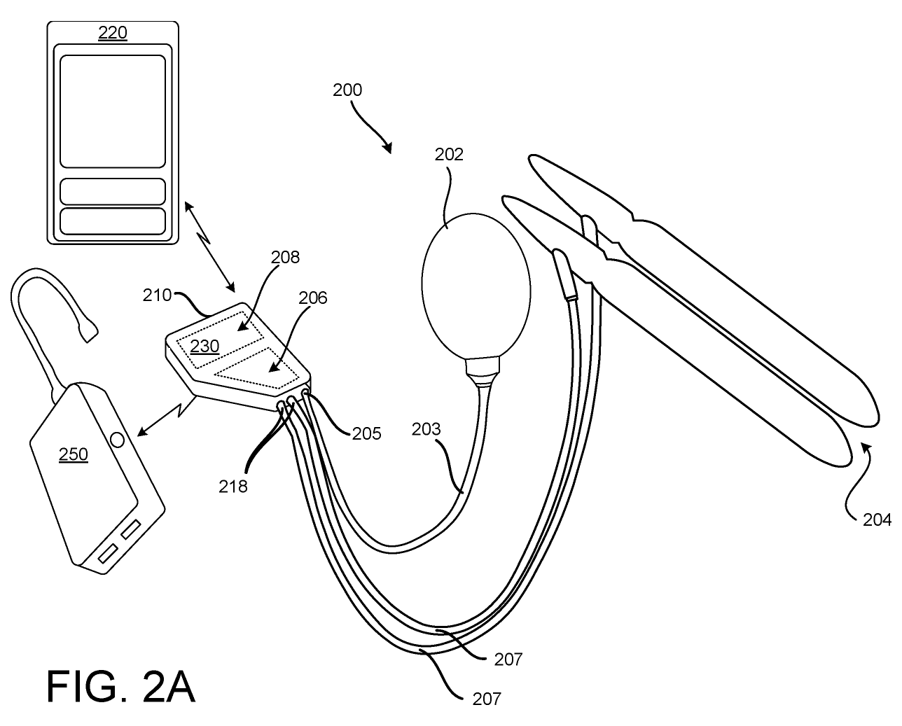

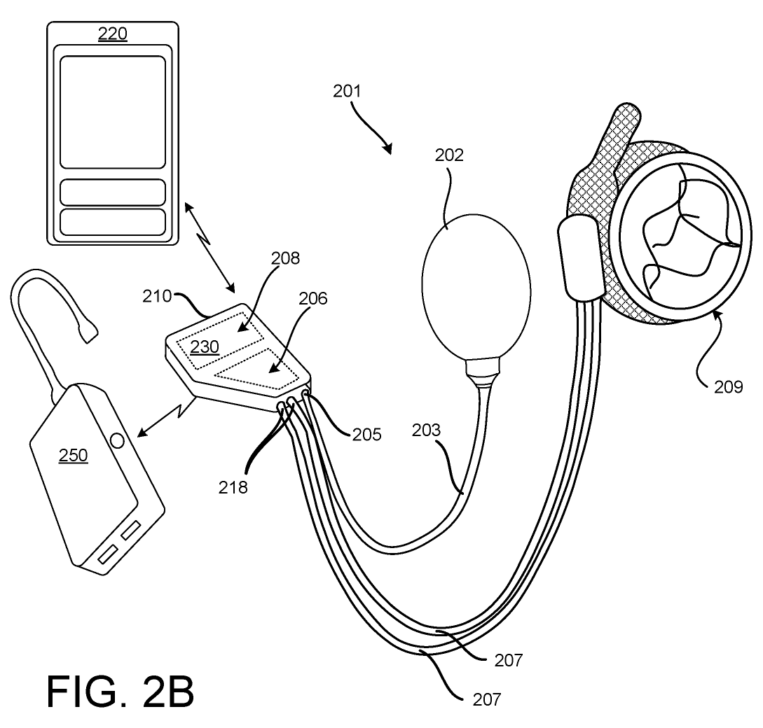

The example implantable inflatable medical device 100 may be representative of a number of different types of implantable fluid-operated devices. For example, the implantable inflatable device 100 shown in FIG. 1 may be representative of an inflatable penile prosthesis as shown in FIG. 2A or an inflatable artificial urinary tract sphincter as shown in FIG. 2B. In some implementations, the example implantable medical device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable inflatable device 200 in the form of an example inflatable penile prosthesis is shown in FIG. 2A. Another example system including an example implantable inflatable device 201 in the form of an example artificial urinary tract sphincter is shown in FIG. 2B. The example implantable inflatable device 200 includes a fluid control system 206 including fluidics components such as pumps, valves, sensing devices and the like positioned in fluid passageways. In some implementations, the fluid control system includes components such as, for example, one or more fluid control devices, one or more pressure sensors, and other such components. In some implementations, the example implantable inflatable device 200 includes an electronic control system 208 configured to provide for the transfer of fluid between a reservoir 202 and an inflatable member 204 via the fluidics components. In the example shown in FIG. 2A, the inflatable member 204 is in the form of a pair of inflatable cylinders, which are configured for implantation within the penis of a patient. In the example shown in FIG. 2B, the inflatable member 209 is in the form of an inflatable cuff that is configured for implantation around the urethra of a patient. In the examples shown in FIGS. 2A and 2B, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204 or the inflatable member 209.

In the example shown in FIG. 2A, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 218 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220, via respective communication modules. For example, an application

stored in a memory and executed by a processor of the external controller 220 may allow the user and/or a physician to operate, view, monitor and alter operation of the implantable inflatable device 200. In some examples, components of the electronic control system 208 and/or the fluid control system 206 can be charged and/or recharged by an energy transmission module of the external controller 220, and/or by an energy transmission device 250, that is separate from the external controller 220. The example implantable inflatable device 200 shown in FIG. 2A includes an electronic control system 208 to provide for control of the operation of the respective inflatable members 204 in the form of cylinders, and the monitoring and control of pressure and/or fluid flow through inflatable members 204.

The principles to be described herein are applicable to the example implantable inflatable device, in the form of the example inflatable penile prostheses shown in FIG. 2A, and to other types of implantable inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. For example, as shown in FIG. 2B, the inflatable member 209 can include an inflatable cuff, which may be implemented as an artificial urinary sphincter. The inflatable cuff 209 is or may be disposed about a urethra proximate the bladder. The implantable inflatable device 201 can be activated to pump fluid from a reservoir to expand the cuff 209 and to close the urethra. The cuff 209 is deflated to allow a patient to void the bladder.

As noted above, the electronic control system 208 controlling the flow of fluid between the reservoir 202 and the inflatable member 204 for inflation, pressurization, deflation, depressurization and the like of the inflatable member 204 may provide for improved patient control of the implantable inflatable device 200, improved accuracy in operation of the implantable inflatable device 200, improved patient comfort, improved patient safety, and the like. In some situations, this improved control and improved accuracy in the operation of the implantable inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or one or more valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

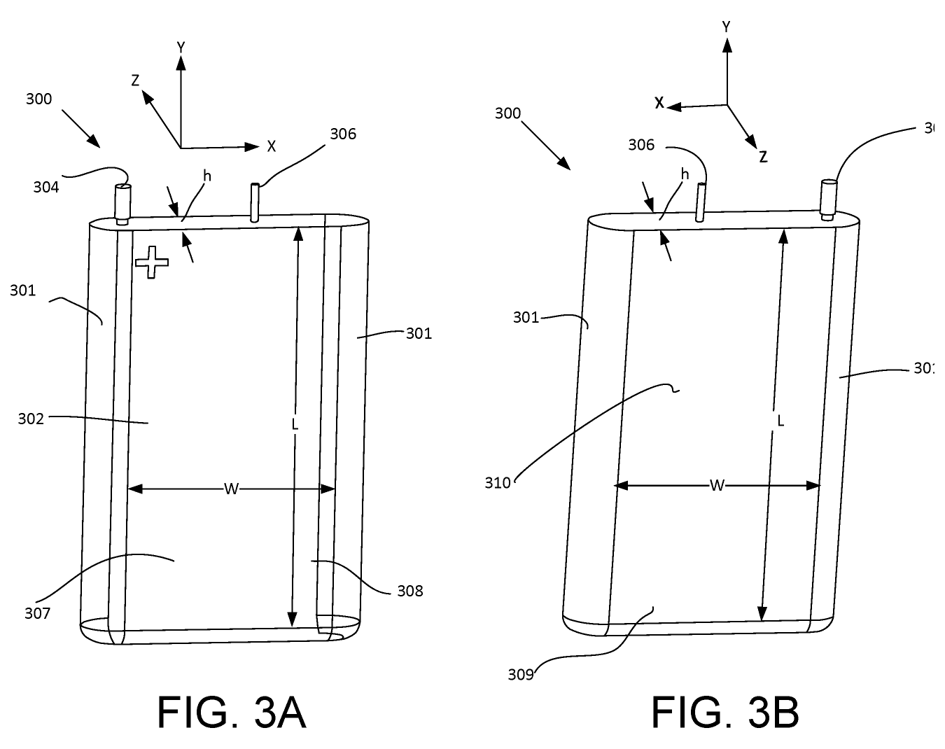

FIG. 3A is a front perspective view of a rechargeable battery 300 that can be used to power electronic components of an implantable medical device, and FIG. 3B is a rear perspective view of the rechargeable battery 300. The rechargeable battery can include various different battery technologies for storing electrical energy, for example, lithium-ion technology, lithium polymer technology, etc. The rechargeable battery 300 can have a form factor of a prismatic cell that has a generally rectangular shape. Although the rechargeable battery 300 as a generally rectangular shape, one or more edges, or sides, 301 of the battery can be rounded. The rechargeable battery 300 can have an outer case 302 that can be made of a flexible metal or polymer material, and lithium prismatic cells can be included within the case, often in a stackable design. The rechargeable battery 300 can include one or more electrodes 304, 306 that can provide energy from the battery to power one or more electronic components.

A Cartesian coordinate system is shown in both FIG. 3A and FIG. 3B. The rechargeable battery 300 can include a length, L, along the y-axis of the coordinate system, a width, W, along the x-axis of the coordinate system and a height, or thickness, h, along the z-axis of the coordinate system. The rechargeable battery can have a first side 307 having a first substantially planar surface 308 and a second side 309 having a second substantially planar surface 310 that is substantially parallel to the first surface 308. In some implementations, the first side 307 and the second side 309 can lie in an X-Y plane of the battery, i.e., extending along the length and width of the battery. In some implementations, the first side 307 and the second side 309 can include substantially planar surfaces, 308, 310, respectively, where the planar surfaces are located within a plane, within a 2% root mean square deviation from planarity. However, as explained herein, in some cases, chemical processes within the battery 300 can cause the geometry of the first side 307 and the second side 309 to deviate from planarity during certain conditions of the use of the battery.

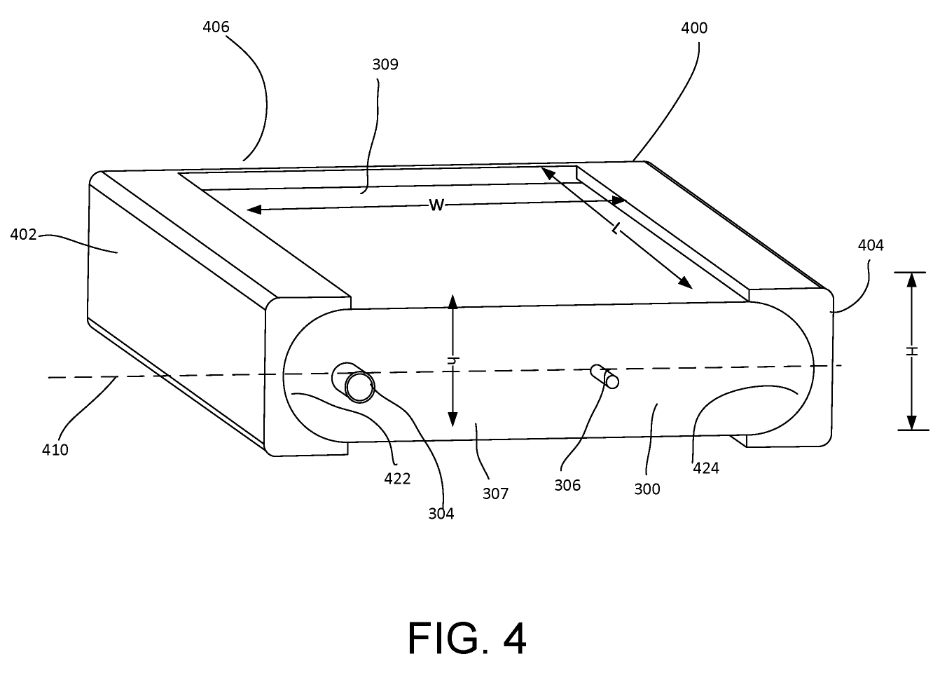

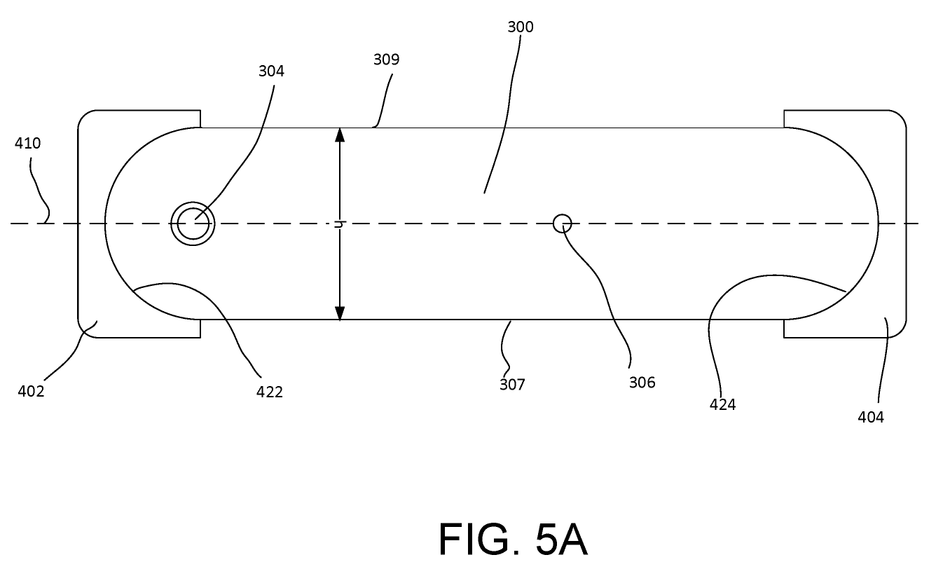

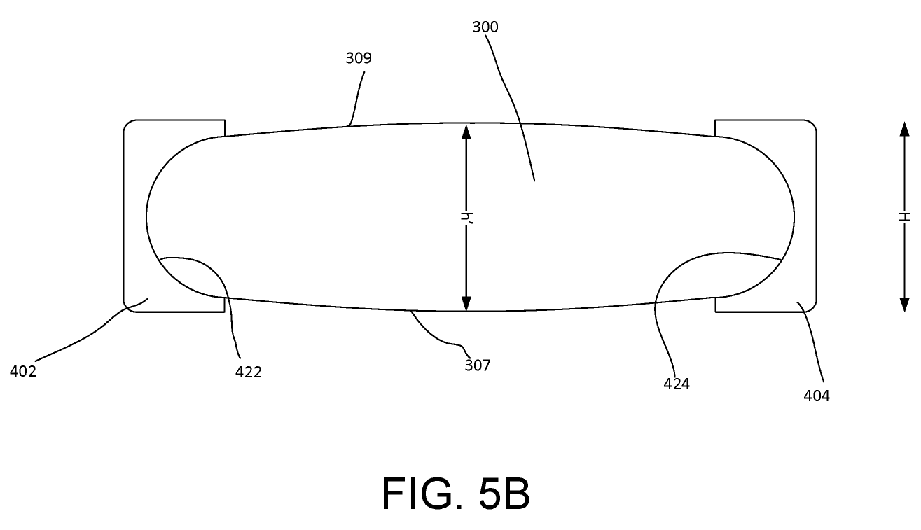

FIG. 4 is a schematic perspective view of the rechargeable battery 300 when it is received and secured by a liner 400. FIG. 5A is a schematic end view of the rechargeable battery 300 when it is received and secured by a liner 400 when the battery is in a normal, unpressurized state. FIG. 5B is a schematic end view of the rechargeable battery 300 when it is received and secured by a liner 400 when the battery is in an unpressurized state, while the battery is being recharged or just after it has been recharged.

In some cases, when the battery 300 is charged, chemical processes within the battery, for example, the emission of gases within the battery as a result of the charging, can cause pressure within the case of the battery to increase in the case to swell in size and to change its shape. If a side of the battery, for example, the first or second side 307, 309 of the battery, were attached directly to a housing or circuit board of the implantable medical device, the change in size and/or shape of the battery could cause the electrodes, 304, 306 of the battery to move relative to the housing or circuit board, which could cause the mechanical and/or electrical connection between the electrodes 304, 306 and the circuit board to be degraded or broken. To avoid this, the liner 400 can be configured for securing the rechargeable battery to a housing of an implantable medical device in a manner in which the case of the rechargeable battery is prevented from contacting the housing of the implantable medical device, so that when the shape and/or size of the rechargeable battery changes in response to charging up the battery the electrodes of the battery do not change their position relative to the housing or circuit board.

In some implementations, the liner 400 includes a first perimeter portion 402 that is configured for receiving a first edge portion 422 of the case at the battery 300. For example, the first perimeter portion 402 can receive the first edge portion 422 along a length, L, of the case of the battery 300. In some implementations, the liner 400 includes a second perimeter portion 404 that is configured for receiving a second edge portion 424 of the case at the battery 300. For example, the second perimeter portion 404 can receive the second edge portion along a length, L, of the case of the battery 300. In some implementations, the liner 400 includes a third perimeter portion 406 that is configured for receiving a third edge portion of the case at the battery 300. For example, the third perimeter portion 406 can receive the third edge portion along a width, W, of the case of the battery 300. The third perimeter portion 406 can be attached to the first perimeter portion 402 into the second perimeter portion 404.

In some implementations, a height, H, of the liner (e.g., a height of the first, second, and third perimeter portions 402, 404, 406 of the liner 400) can be greater than the height, h, of the case of the battery 300. For example, the height, H, of the liner can be more than the maximum distance, h’, between the first side 307 and the second side 309 of the battery, which is attained in response to the battery being recharged. In another implementation, the height of the liner can be at least 10% greater than the height of the case when the case is not being recharged and is in an undeformed shape with the first side 307 being substantially parallel to the second side 309. In some implementations, the first perimeter portion 402 and the second perimeter portion 404 of the liner do not contact the first substantially planar surface 308 or the second substantially planar surface 310 of the battery 300 when the battery is received within the liner, but rather the first perimeter portion 402 contacts a curved edge portion 422 of the battery case, and the second perimeter portion 404 contact a curved edge portion 424 of the battery case.

In some implementations, the electrodes 304, 306 of the battery 300 can extend from an end of the battery case. In some implementations, the electrodes can be located along a midplane 410 of the battery case, which is midway between the first side 307 and the second side 309 of the case. With the electrodes 304, 306 located on the midplane 410, when the battery 300 is received and secured within the liner 400 and the battery case swells and changes shape in response to the battery being charged, the position of the electrodes 304, 306 can be unchanged in the vertical direction perpendicular to the midplane 410.

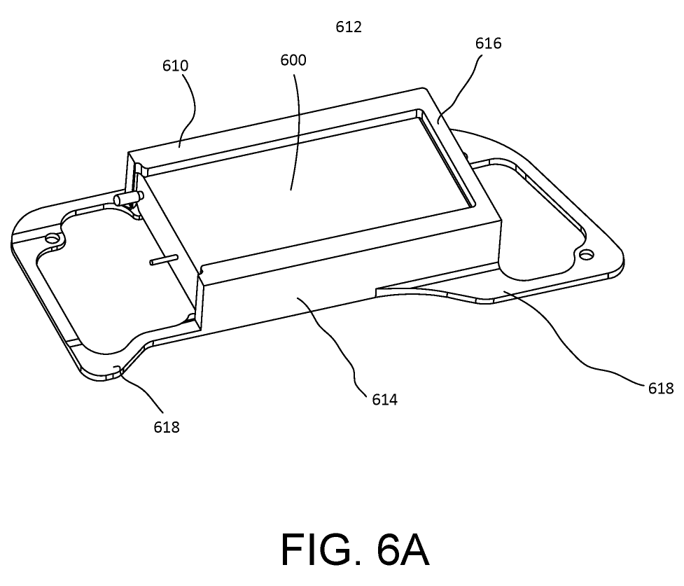

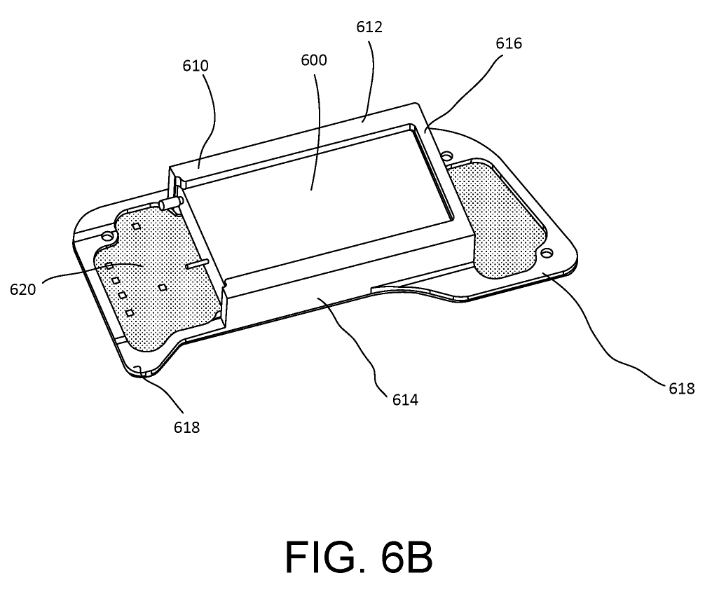

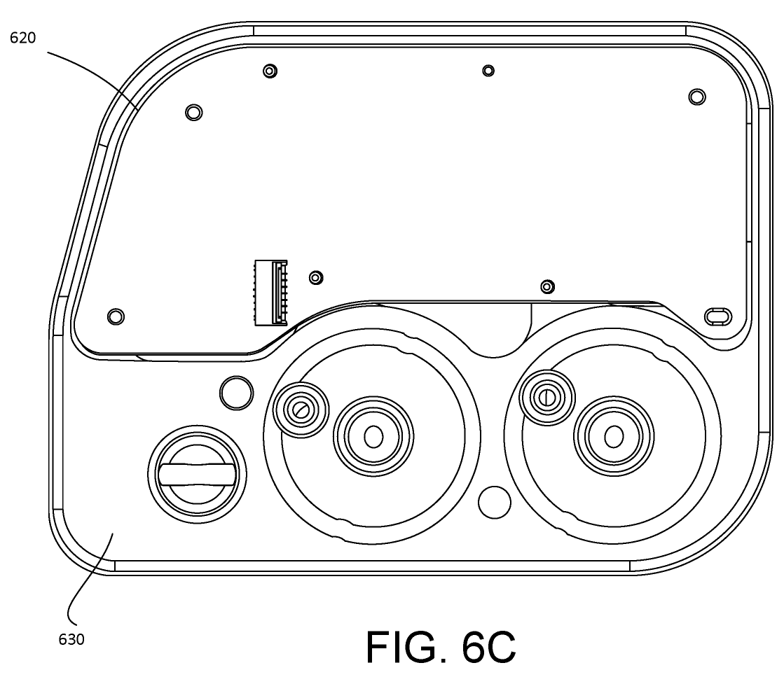

FIG. 6A is a schematic perspective view of a battery 600 received and secured within a battery liner 610. FIG. 6B is a schematic perspective view of the battery 600 received and secured within the battery liner 610, with a printed circuit board being received and attached to the liner. FIG. 6C is a schematic bottom view of the printed circuit board 620 attached to a housing 630 of an electronic device, where the housing can include a fluidic manifold configured for pumping fluid between a reservoir and an inflatable member. The battery 600 can be received and secured within the battery liner 610, with a printed circuit board being received and attached to the liner. The battery liner 610 can include a first perimeter portion 612, a second perimeter portion 614, and a third perimeter portion 616, which receive and secure edges a case of the battery 600. In addition, the battery liner 610 can include a fourth perimeter portion 618 that is configured for receiving one or more printed circuit boards 620, where the one or more printed circuit boards include at least one electrical component that is powered by the battery 600. The fourth perimeter portion 618 of the liner 610 can be attached to at least one of the first perimeter portion 612, the second perimeter portion 614 and/or the third perimeter portion 616 of the liner 610.

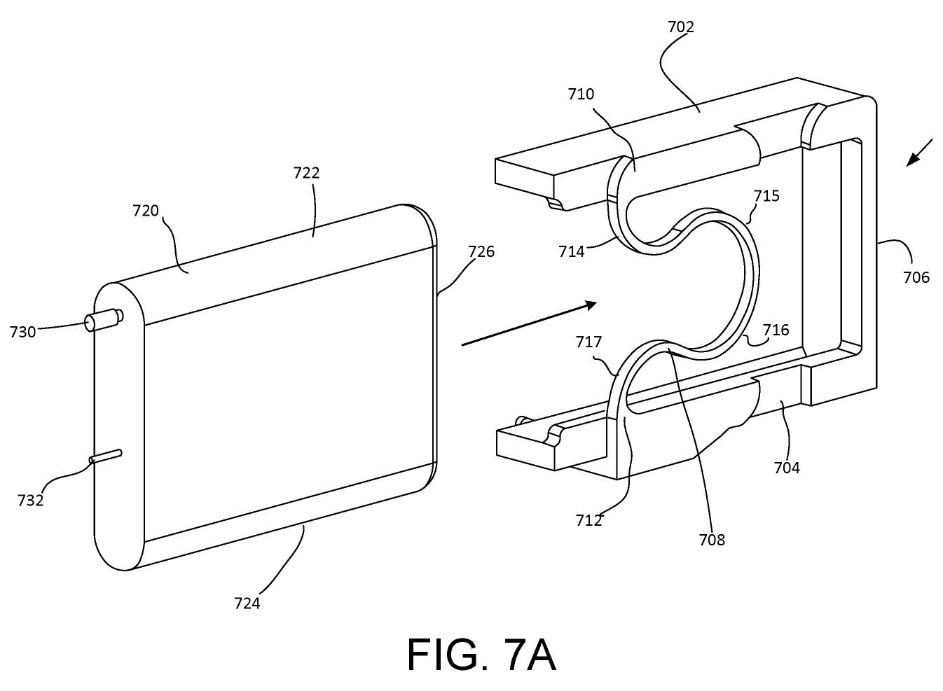

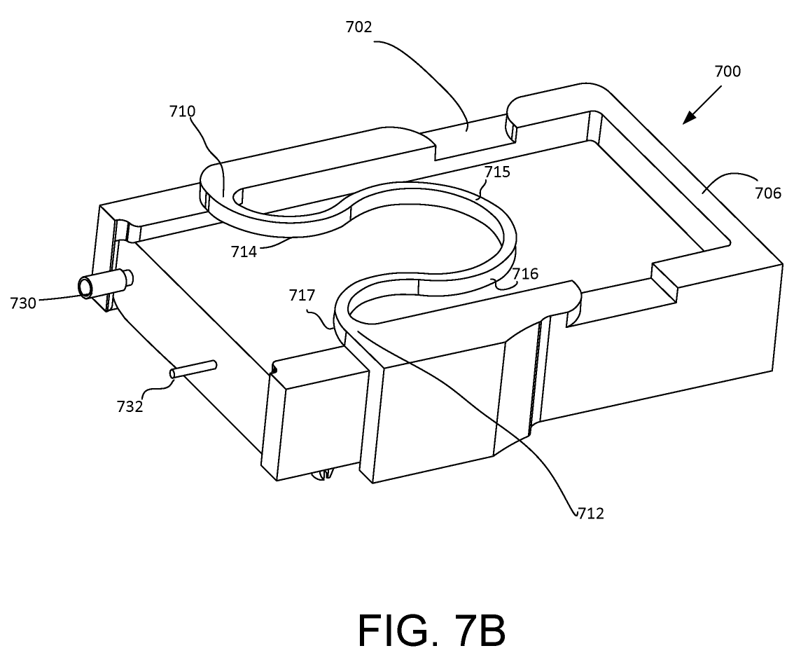

FIG. 7A is a schematic perspective view of a liner 700 that is configured for receiving a battery 720. FIG. 7B is a schematic perspective view of the liner 700 that has received and secured the battery 720. As shown in FIG. 7A and in FIG. 7B, a unitary structure that includes a first perimeter portion that receives a first edge 722 of the battery, a second perimeter portion 704 that receives a second edge 724 of the battery and a third perimeter portion 706 that receives a third edge 726 of the battery, where the third perimeter portion 706 connects, and is attached to, the first and third perimeter portions 702, 704. In addition, the unitary structure of the liner 700 can include a curved retaining band 708 that is attached to locations 710, 712 of the first and second perimeter portions 702, 704, respectively, which are distal from the third perimeter portion 706. The curved retaining band 708 can have a distance along its length that is greater than the distance between the locations 710, 712. In some implementations, the curved retaining band 708 can have a geometry in which a segment 714 of the band extends away from the first perimeter portion 702 and then curves toward the first perimeter portion, a segment 715 that curves away from the first perimeter portion, a segment 716 that curves toward the first perimeter portion, and a segment 717 that curves away from the first perimeter portion and terminates happy second perimeter portion 704.

In some implementations, the liner 700 can be formed (e.g., by a molding process, for example, by an injection molding process) into a unitary structure, and the material of the liner can include a polymer material. The material of the liner 700 can be flexible, so that perimeter portions 702, 704 can be manipulated by hand into different distances from each other. For example, the first and second perimeter portions 702, 704 can be pulled apart from each other, so that the distance between locations 710, 712 increases to allow the battery 720 slide into position within the liner 700. The curved retaining band 708 can act as a spring to allow the first and second perimeter portions 702, 704 to be pulled apart from each other but to exert a force that pulls the first and second perimeter portions toward each other once the force that pulls the perimeter portions of art is removed.

The battery 720 can be slid into position within the liner 700 with the electrodes 730, 732 of the battery facing outward and away from the liner and with the electrodes located distal from the third perimeter portion 706 of the liner.

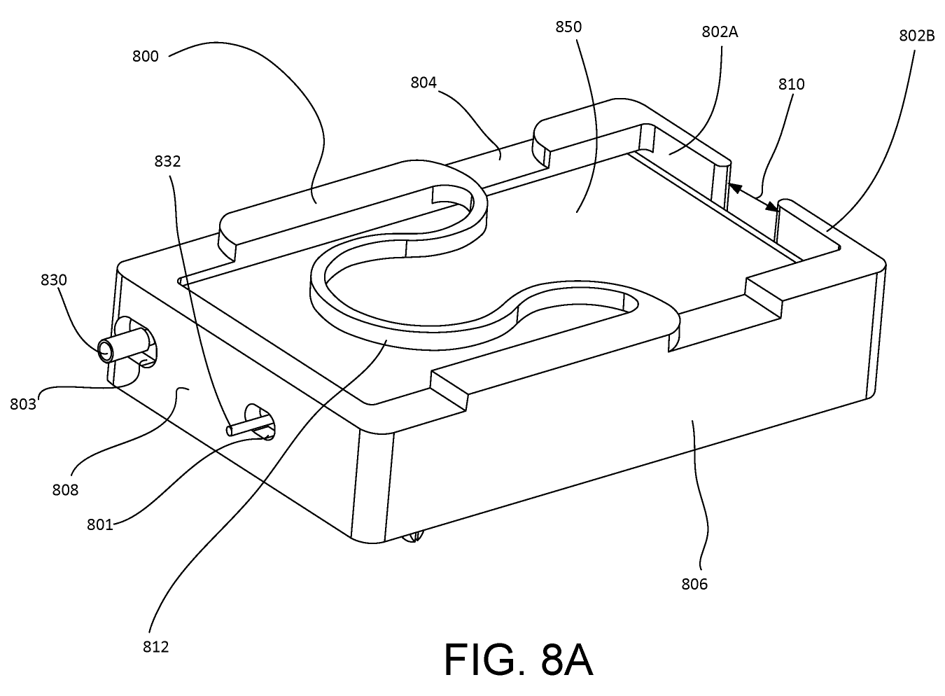

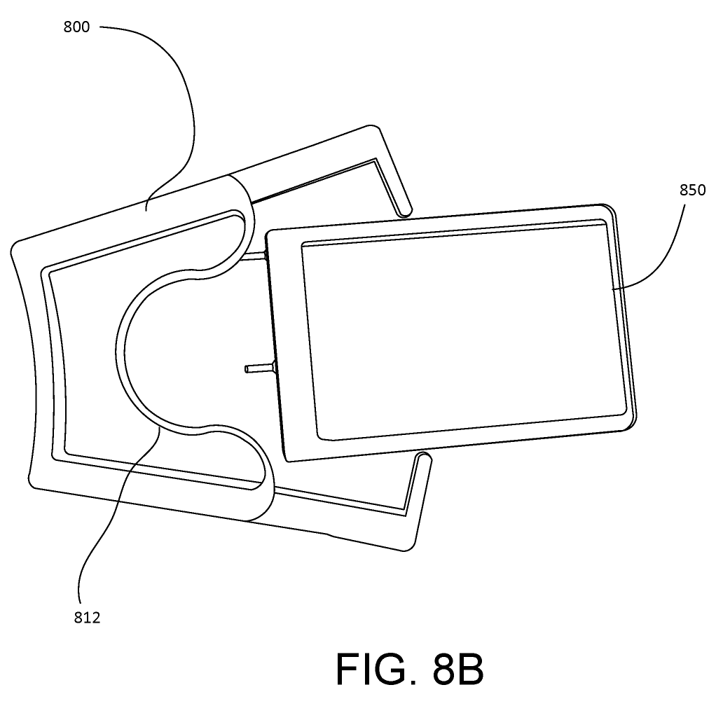

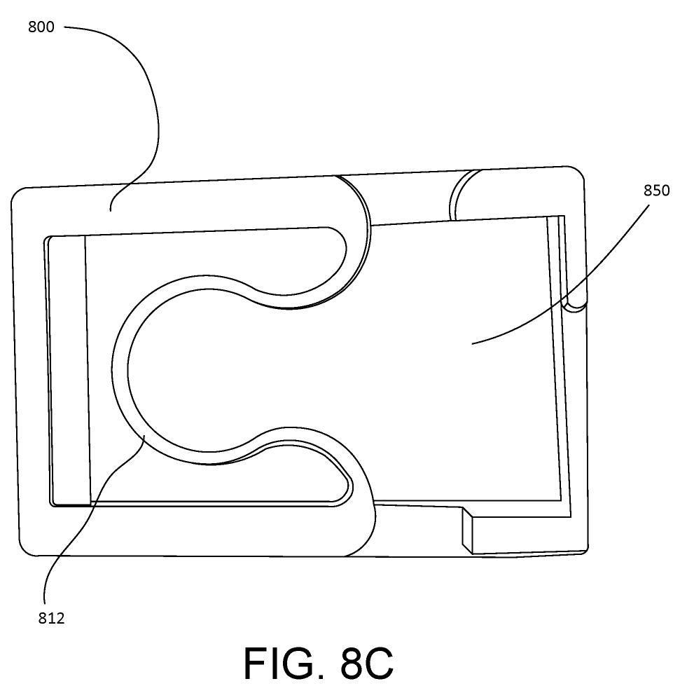

FIG. 8A is a schematic perspective view of a liner 800 that is configured for receiving a battery 850. FIG. 8B is a schematic top view of the liner 800 that is receiving the battery 850. FIG. 8C is a schematic top view of the liner 800 that has received the battery 850. The liner 800 is similar to the liner 700 of FIGS. 7A and 7B, but with the liner 800 including the additional features that are not included in the liner 700. For example, the liner 800 includes a first perimeter portion 804, a second perimeter portion 806, and a third perimeter portion 808 that are configured to receive and secure first, second, and third edge portions of the case of the battery 850. In addition, the battery liner 800 includes a fourth perimeter portions 802A and 802B that receives and secures a fourth edge portion 804 of the battery.

In some implementations, a gap 810 can exist between the fourth perimeter portions 802A and 802B either or both when the battery 850 is being received into the liner 800 and when the battery has been received and secured within the liner. Ends of the first and second perimeter portions, which are attached to the perimeter portions 802A and 802B, respectively, can be forced apart from each other (e.g., as shown in FIG. 8B), so that the battery 850 can be inserted into the battery liner 800. Then, once the battery 850 has been received within the liner 800, a curved retaining band 812 that is part of the liner 800 can pull the first and second perimeter portions 804, 806 toward each other to secure the battery 850 within the liner 800.

In addition, the third perimeter portion 806 of the liner 800 can include openings 801 and 803 that are configured to permit electrodes 830, 832 of the battery to pass through, and protrude away from, the third perimeter portion. Therefore, the battery 850 can be inserted into the liner 800 with the electrodes 830, 832 facing into the liner (e.g., as shown in FIG. 8B), and then once the battery is secured within the liner, the electrodes 830, 832 can pass through, and protrude away from, the third perimeter portion 808 of the liner 800.

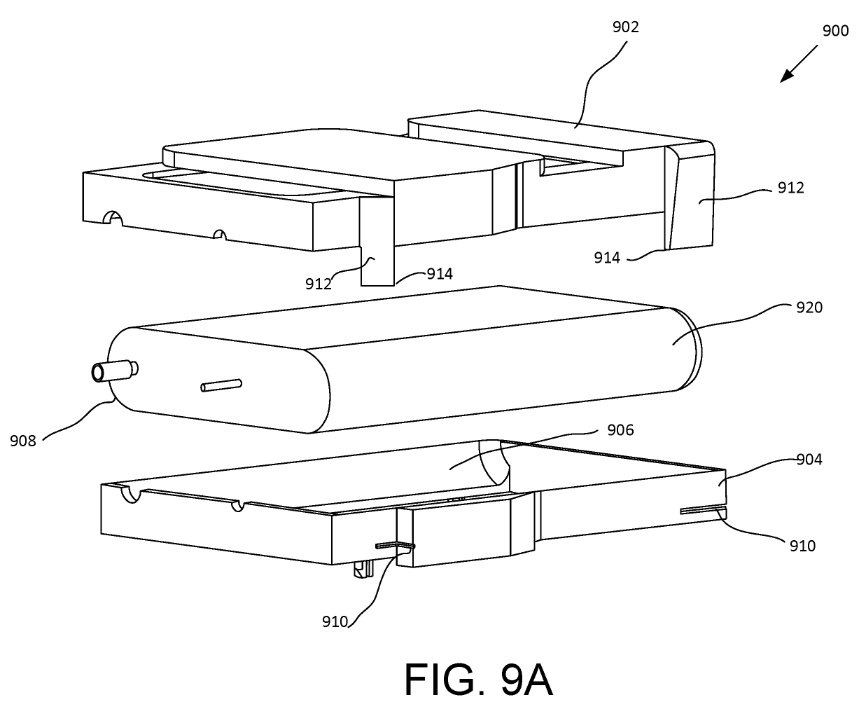

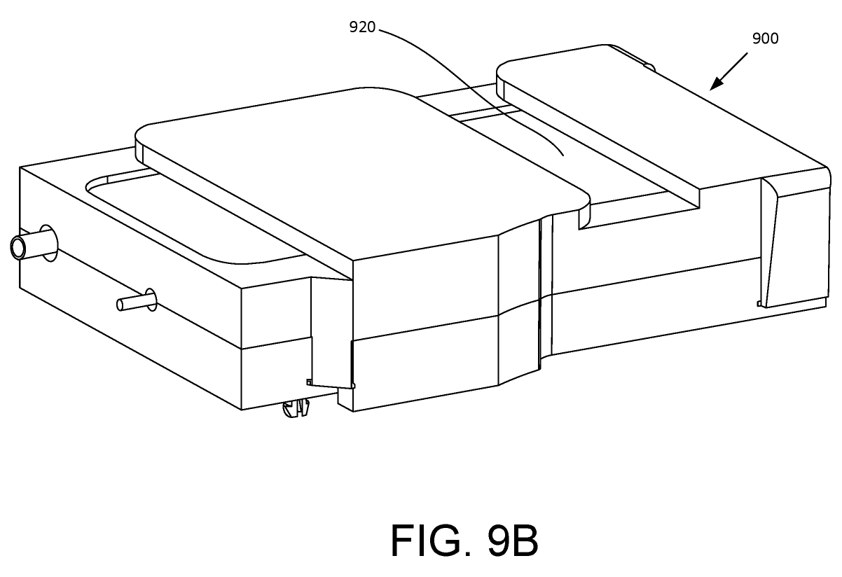

FIG. 9A is a schematic exploded view of a multi-part liner 900 that is configured for receiving a battery 920. FIG. 9B is a schematic perspective view of the liner 900 that has received the battery 920. The liner 900 can include a first, top part 902 and the second, bottom part 904, where the two parts 902, 904 can cooperate to receive and secure a battery 920. In some implementations, the top part and the bottom part 902, 904 can include perimeter portions that are shaped to conform to edge portions of the battery 920. For example, a section 906 of a perimeter portion of the bottom part 904 can include an interior side wall that is curved to conform to a curved edge 908 of the battery. Other sections of perimeter portions of the bottom part 904 and the top part 902 can be shaped to similarly conform to the shape of the edge portions of the battery 920.

The battery 920 can be placed into the bottom part 904 or the top part 902, and then the other part can be fastened to the part in which the battery has been placed to secure the battery within the multi-part liner 900. For example, once the battery 920 has been placed within the bottom part 904, the top part 902 can be coupled to the bottom part to enclose and secure the battery within the multi-part liner 900. The top part 902 and the bottom part 904 can be coupled to each other in a variety of ways. For example, in one implementation, one part can include one or more grooves or slots 910 and the other part can include one or more respective tabs 912 having protrusions 914 that extend from the tabs 912 and that are configured to fit into the respective groups or slots 910. Thus, when the top part 902 includes the tabs 912 and the bottom part includes the slots 910 and the battery 920 is in the bottom part, the top part can be manipulated toward the bottom part from above until the protrusions 914 from the tabs 912 coupled with the grooves 910 to secure the top part to the bottom part. In other implementations, the top part and two and the bottom part 904 can be coupled to each other by, for example, adhesive material, fasteners (e.g., screws, rivets, etc.), etc.

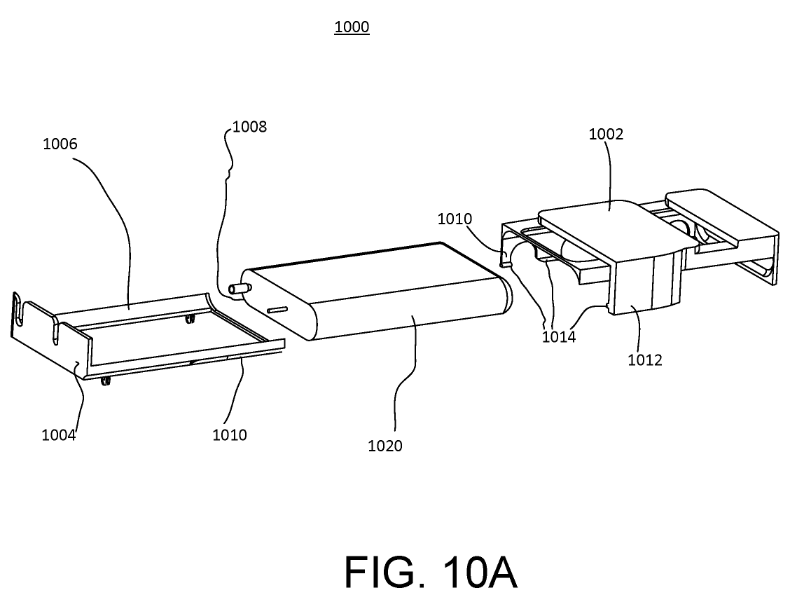

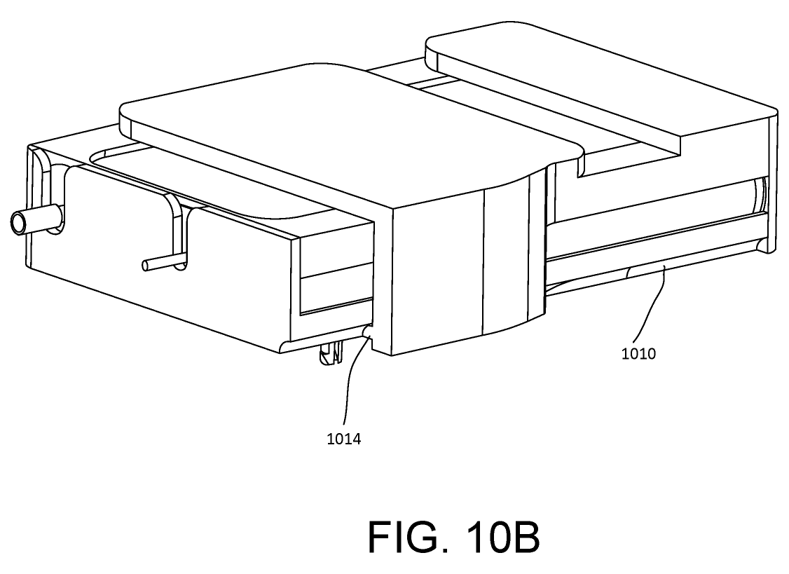

FIG. 10A is a schematic exploded view of another multi-part liner 1000 that is configured for receiving a battery 1020. FIG. 10B is a schematic perspective view of the liner 1000 that has received the battery 1020. The liner 1000 can include a first, top part 1002 and the second, bottom part 1004, where the two parts 1002, 1004 can cooperate to receive and secure a battery 1020. In some implementations, the top part and the bottom part 1002, 1004 can include perimeter portions that are shaped to conform to edge portions of the battery 1020. For example, a section 1006 of a perimeter portion of the bottom part 1004 can include an interior side wall that is curved to conform to a curved edge 1008 of the battery. Other sections of perimeter portions of the bottom part 1004 and the top part 1002 can be shaped to conform similarly to the shape of the edge portions of the battery 1020.

The battery 1020 can be placed into the bottom part 1004 or the top part 1002, and then the other part can be fastened to the part in which the battery has been placed to secure the battery within the multi-part liner 1000. For example, once the battery 1020 has been placed within the bottom part 1004, the top part 1002 can be coupled to the bottom part to enclose and secure the battery within the multi-part liner 1000. The top part 1002 and the bottom part 1004 can be coupled to each other in a variety of ways. For example, in one implementation, one part can include one or more grooves or slots 1010 and the other part can include rails 1014, which, in some implementations, can extend from tabs 1012 of the other part, where the rails are configured to fit into the respective groups or slots 1010. Thus, when the top part 1002 includes the rails 1014 and the bottom part includes the slots 1010 and the battery 1020 is in the bottom part, the rails 1014 of the top part can slide into the grooves 1010 of the bottom part to secure the top part to the bottom part.

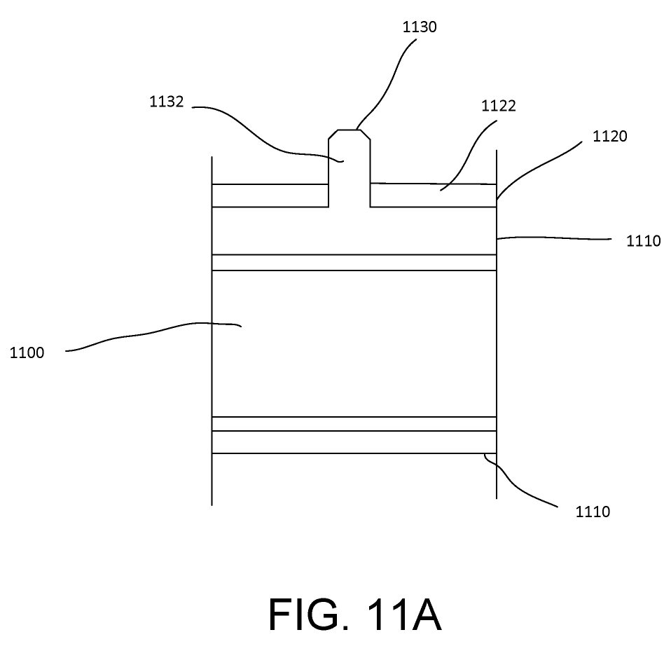

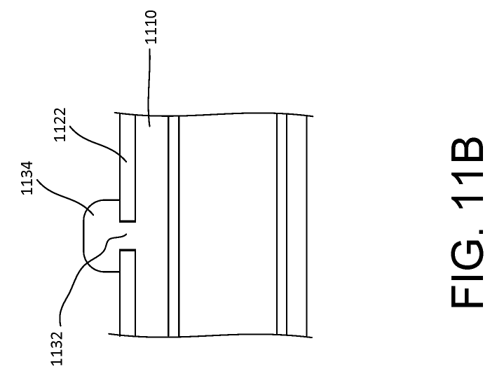

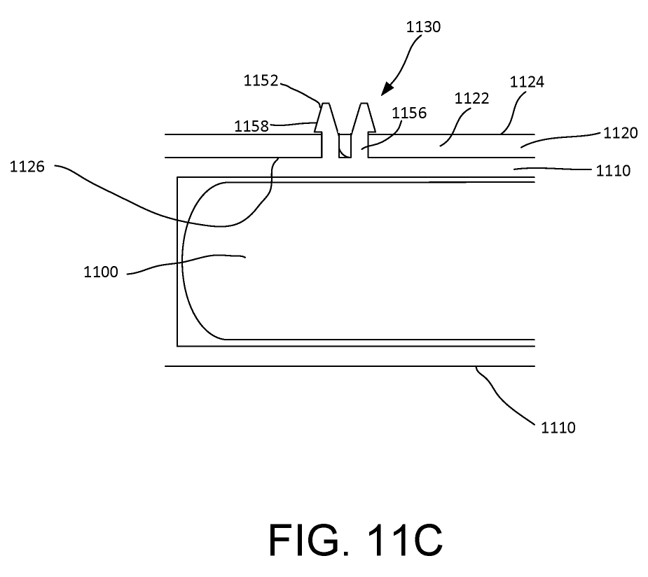

With the battery secured with the liner, the liner can be securely attached to a housing of an implantable medical device. FIG. 11A is an example partial side view of a battery 1100 disposed within a liner 1110 with the liner being attached to a housing 1120 of an implantable medical device by a coupling member 1130. The coupling member 1130 includes a post 1132 that is integral to and extends from a side of the liner 1110. In some examples, the post 1132 includes a cylindrical post defining a diameter. In some examples, the post’s diameter is less than or equal to a diameter of a hole in a wall 1122 of the housing 1120 to which the liner 1110 is coupled. In some implementations, the post 1132 extends through and out of the hole and can have a length that is greater than the thickness of the wall 1122, such that the post 1132 can include a portion that extends out of the hole in the wall 1122. In some implementations, the post is not extended through and out of the hole that has a length that is shorter than the thickness of the wall 1122. In some implementations, the post 1132 can have a diameter relative to the diameter of the hole in the wall 1122, such that the post can be press fit into the hole to secure the liner 1110 to the housing 1120. In some implementations, adhesive can be applied to one or more of the post 1132 and interior walls of the hole to secure the liner 1110 to the housing 1120.

FIG. 11B is another example partial side view of the battery 1100 disposed within the liner 1110 with the liner being attached to the housing 1120 of the implantable medical device by the coupling member 1130. As shown in FIG. 11B, after the post 1132 has been inserted through the hole in the wall 1122 of the housing 1120, the post 1132 can be secured to the wall by expanding an effective diameter, d, of a portion 1134 of the post above the wall 1122. In some implementations, a quantity of adhesive can be applied to the portion of the post 1132 that extends above the wall 1122 to expand the effective diameter of the post and to secure the liner 1110 to the housing 1120. The adhesive may be an encapsulation (e.g., a high-pressure injection molded material), glue, epoxy, or other adhesive that can be dispensed or molded over the top of the post 1132. The adhesive may adhere to both the post 1132 and the circuit substrate wall 1122, which may prevent (or reduce) motion of the liner and battery with respect to the housing. In some implementations, the material of the post 1132 can be melted to create the portion 1134 that has a diameter greater than the diameter of the hole in the wall 1122 of the housing.

FIG. 11C is another example partial side view of the battery 1100 disposed within the liner 1110 with the liner being attached to the housing 1120 of the implantable medical device by the coupling member 1130. As shown in FIG. 11C, the coupling member 1130 includes a snap-fit rivet 1152. The rivet 1152 may be positioned in the hole of the wall 1122. The rivet 1152 can include one or more features that contact the wall 1122 to secure the liner 1110 to the housing 1120. The rivet 1152 can be formed of two parts that allow the rivet to snap-fit into the hole. Each of the two parts of the rivet 1152 can include a neck portion 1156, and a flared portion 1158. A diameter of the rivet can be defined along the length of the rivet as the distance between outermost portions of the two parts of the rivet in a plane perpendicular to the length of the rivet.

The neck portion 1156 may have a size (e.g., a diameter) that is smaller than the size (e.g., diameter) of the hole in the wall 1122. In some examples, the flared portion 1158 can have a diameter that varies along its length from a minimum diameter at the end of the rivet to a maximum diameter at the end of the flared portion that is distal from the end of the rivet. In some examples, the flared portion the rivet 1152 can be pressed through the hole from a bottom side 1126 of the wall 1122, causing the two parts of the rivet to move towards each other as the forces exerted by the sidewalls of the hole on the flared portion of the rivet, causing the neck portions of the rivet to bend toward each other, thus reducing a diameter of the flared portion to allow the flared portion to fit through the hole. Once the flared portion 1158 of the rivet 1130 is passed through the hole in the wall 1122, the neck portions of the rivet revert to their unstressed state such that the diameter of the flared portion increases and locks the liner into contact with a top side 1124 of the wall 1122 of the housing.

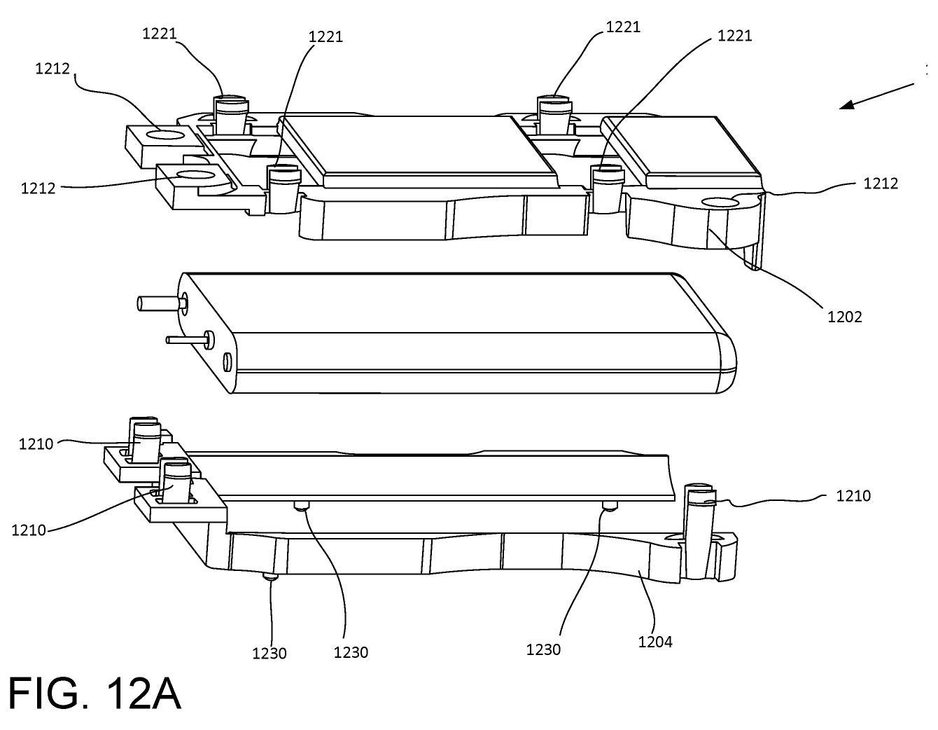

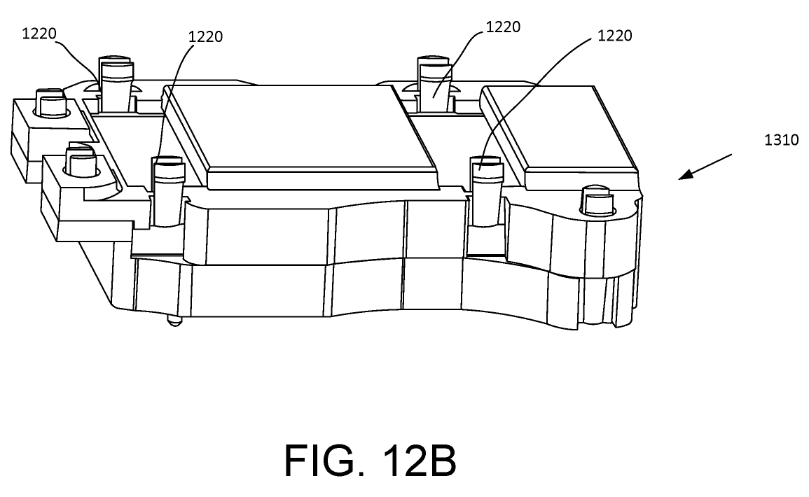

FIG. 12A is a schematic exploded view of another multi-part liner 1200 that is configured for receiving a battery 1220 and for attaching to a housing of an implantable medical device. FIG. 12B is a schematic perspective view of the liner 1200 that has received the battery 1220. The multi-part liner 1200 is similar to the multi-part liner 900 of FIG. 9A and FIG. 9B, and that it includes a top part 1202 and a bottom part 1204 that are configured for coupling to each other to enclose the battery 1220 within the liner. The top part 1202 can be coupled to the bottom part 1204 by way of a first plurality of coupling members 1210 included in one part that couple to a first plurality of holes 1212 in the other part. As shown in FIGS. 12A and 12B, the coupling members 1210 can be included in the bottom part 1204 and the holes can be included in the top part, but the reverse is also contemplated. The coupling members can include, for example, posts, rivet, fasteners, etc. Additionally, the liner 1200 can include a second plurality of coupling members 1221 that are configured for coupling the lining 1200 to a housing of the implantable medical device. Furthermore, the liner 1200 can include a third plurality of coupling members 1230 that are configured for coupling the lining 1200 to a printed circuit board of the implantable medical device.

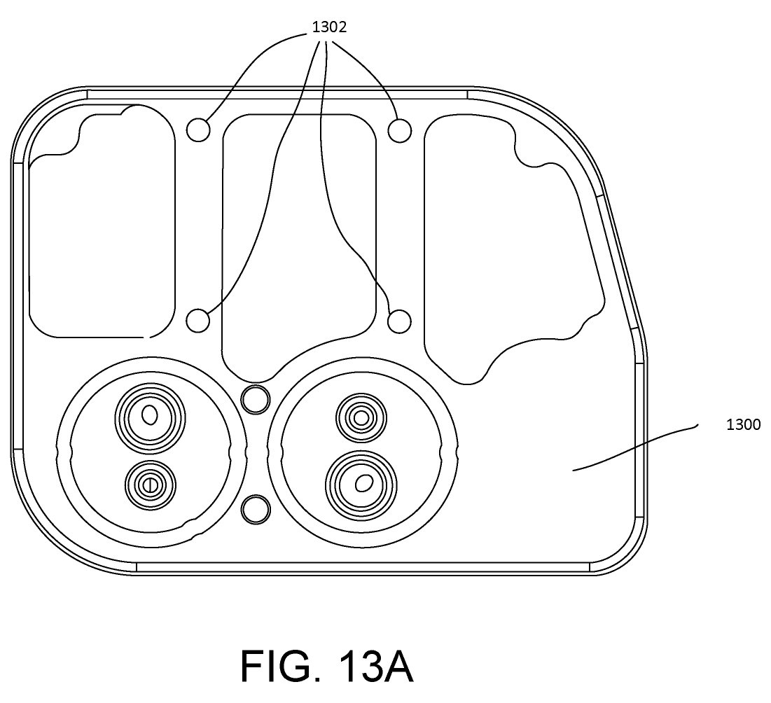

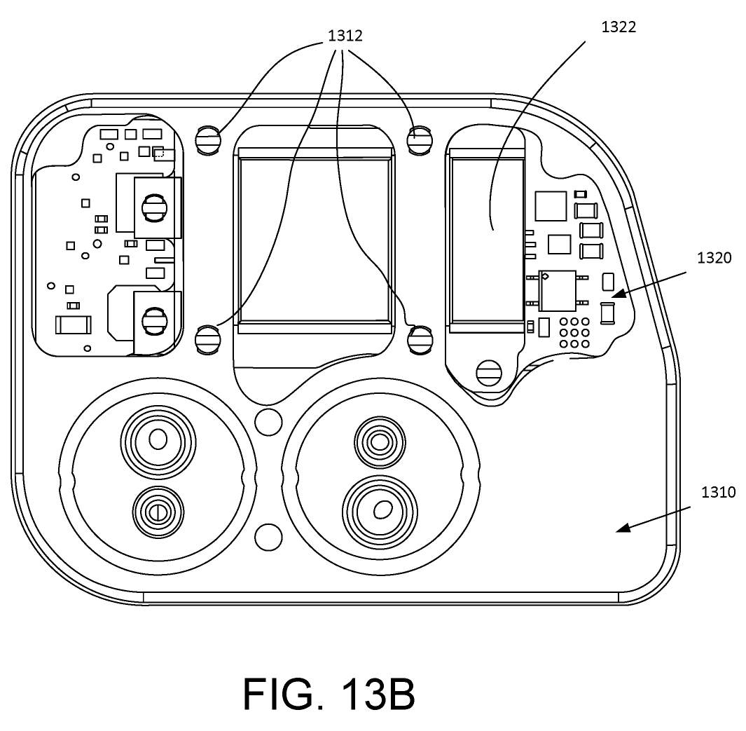

FIG. 13A is a schematic bottom view of a housing 1300 of an implantable medical device. The housing can include a manifold that includes, or that is configured to received, one or more electronic fluidic pumps, and which is configured for pumping fluid between a reservoir and an inflatable member. FIG. 13B is a schematic bottom view of the housing 1300 with a battery liner 1310, which is attached to a printed circuit board 1320, secured to the housing 1300. The housing 1300 can include a plurality of holes 1302 that are configured for receiving and coupling to coupling members 1312 that are part of the liner 1310. As shown in FIG. 13B, the coupling members 1312 of the liner coupled to the holes 1302 of the housing to secure the liner to the housing.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir;
an electronic pump device configured to transfer fluid between the inflatable member and the fluid reservoir, the electronic pump device including a housing;
a rechargeable battery that includes a case and at least one electrode, the rechargeable battery being configured to supply electrical power to the electronic pump device, wherein a shape and a size of the rechargeable battery changes in response to the rechargeable battery being recharged; and
a liner configured for securing the rechargeable battery to the housing of the electronic pump device and for preventing the case of the rechargeable battery from contacting the housing of the electronic pump device when the shape and size of the rechargeable battery changes in response to the rechargeable battery being recharged.

2. The implantable medical device of claim 1, wherein the case of the rechargeable battery includes a first substantially planer surface and a second substantially planar surface that is parallel to the first surface, and wherein a maximum distance between the first surface and the second surface increases by more than 10% in response to the rechargeable battery being recharged.

3. The implantable medical device of claim 2, wherein the first substantially planar surface has a first width and a first length and the second substantially planar surface has a second width and a second length, wherein the second width is within 10% of the first width and the second length is within 10% of the first length, and wherein a height of the case between the first substantially planar surface and the second substantially planar surface is less than 50% of the first width.

4. The implantable medical device of claim 3, further comprising one or more electrodes located on a face of the battery defined by the height of the battery and a width that is substantially parallel to the first and second substantially planar surfaces.

5. The implantable medical device of claim 4, wherein the one or more electrodes are located on a midplane between the first and second substantially planar surfaces.

6. The implantable medical device of claim 3, wherein the liner includes a first perimeter portion configured for receiving a first edge portion of the case along a length of the case, a second perimeter portion configured for receiving a second edge portion of the case along a length of the case, and a third perimeter portion configured for receiving a third edge portion of the case along the width of the case, the third perimeter portion being attached to the first and second perimeter portions.

7. The implantable medical device of claim 6, wherein the first, second, and third perimeter portions have a height that is more than the maximum distance between the first surface and the second surface that is attained in response to the rechargeable battery being recharged.

8. The implantable medical device of claim 6, wherein the first, second, and third perimeter portions have a height that is at least 10% greater than the height of the case.

9. The implantable medical device of claim 6, wherein the liner includes no portions that contact the first substantially planar surface or the second substantially planar surface.

10. The implantable medical device of claim 6, wherein the liner further includes a fourth perimeter portion configured for receiving a printed circuit board, wherein the printed circuit board includes at least one electrical component that is powered by the battery.

11. The implantable medical device of claim 6, wherein the liner further includes a fifth perimeter portion configured for receiving a fourth edge portion of the case along the width of the case.

12. The implantable medical device of claim 6, wherein the liner further includes one or more first coupling members that couple the liner to the housing of the electronic pump device.

13. The implantable medical device of claim 12, wherein each of the one or more first coupling members includes a post that is integral to, and extends from, the liner through a respective hole in the housing.

14. The implantable medical device of claim 13, wherein the first coupling members include an adhesive material disposed on the post.

15. The implantable medical device of claim 13, wherein each of the first coupling members includes a rivet.

16. The implantable medical device of claim 15, wherein the rivet includes a head portion, a neck portion, and a flared portion, and the head portion contacts the housing, and the neck portion is disposed in the hole.

17. The implantable medical device of claim 12, wherein the liner further includes one or more second coupling members configured for coupling the liner to a printed circuit board, wherein the printed circuit board includes at least one electrical component that is powered by the battery, wherein each of the second coupling member includes a post that is integral to liner and is configured to extend from the liner through a hole in the printed circuit board.

18. The implantable medical device of claim 17, wherein each of the second coupling members includes an adhesive material disposed on the post.

19. The implantable medical device of claim 17, wherein each of the second coupling members includes a rivet having a head portion, a neck portion, and a flared portion, wherein the head portion is configured to contact the printed circuit board, and the neck portion is configured to be disposed in a hole in the printed circuit board.

20. The implantable medical device of claim 1, wherein the inflatable member includes an inflatable cuff configured for implantation about a urethra of a patient or
a penile implant.
